(19) United States
(12) Patent Application Publication
(10) Pub. No.: US 2024/0226702 A1
(43) Pub. Date: Jul. 11, 2024

(54) PROCESSING SYSTEM, PROCESSING METHOD, AND PROGRAM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Keisuke SUGA, Nagoya-shi (JP)

(73) Assignee: TOYOTA JIDOSHA KABUSHIKI KAISHA, Toyota-shi (JP)

(21) Appl. No.: 18/404,483

(22) Filed: Jan. 4, 2024

(30) Foreign Application Priority Data
Jan. 5, 2023 (JP) 2023-000395

(51) Int. Cl. A63B 71/06; A63B 24/00

(52) U.S. Cl. CPC A63B 71/0616; A63B 24/0062; A63B 24/0075; A63B 71/0622; A63B 2024/0065; A63B 2024/0068

(57) ABSTRACT

A processing system according to an embodiment includes a schedule information acquisition unit configured to acquire schedule information indicating schedules of a plurality of users, an extraction unit configured to extract exercise available time slots from the schedules of the users, and a presentation unit configured to compare the exercise available time slots of the plurality of users and present users who can exercise together and corresponding time slots.

### CROSS REFERENCE TO RELATED APPLICATIONS

This application is based upon and claims the benefit of priority from Japanese patent application No. 2023-000395, filed on Jan. 5, 2023, the disclosure of which is incorporated herein in its entirety by reference.

### BACKGROUND

The present disclosure relates to a processing system, a processing method, and a program.

Japanese Unexamined Patent Application Publication No. 2010-3222 discloses a health support system having an exercise information providing terminal. The exercise information providing terminal provides information such as an exercise program recommended at exercise facilities.

### SUMMARY

In the above system, it may be difficult to provide a user with appropriate motivation to exercise.

The present disclosure has been made to solve such a problem, and provides a processing system, a processing method, and a program capable of appropriately motivating a user to exercise.

A processing system according to an embodiment includes: a schedule information acquisition unit configured to acquire schedule information indicating schedules of a plurality of users; an extraction unit configured to extract exercise available time slots from the schedules of the users; and a presentation unit configured to compare the exercise available time slots of the plurality of users and present users who can exercise together and corresponding time slots.

The above processing system may further include a monitor configured to display the users performing exercises simultaneously.

In the above processing system, the presentation unit may present users whose exercise available time slots match that of another user so that the plurality of users can exercise simultaneously.

In the above processing system, the presentation unit may present users whose exercise available time slots are shifted from that of another user so that the plurality of users can exercise in sequence.

In the above processing system, the presentation unit may provide exercise content according to the exercise available time slots.

In the above processing system, the user information indicating an exercise level or a physical level of the user may be acquired and another user of the same level may be presented.

In the above processing system, the user information indicating an exercise level or a physical level of the user may be acquired and an exercise volume difference may be presented according to a difference in the exercise level and the physical level from those of another user.

A processing method including: acquiring schedule information indicating schedules of a plurality of users; extracting exercise available time slots from the schedules of the users; and comparing the exercise available time slots of the plurality of users and presenting users who can exercise together and corresponding time slots.

The users performing exercises simultaneously may be displayed on a monitor.

The presentation unit may present users whose exercise available time slots match that of another user so that the plurality of users can exercise simultaneously.

The presentation unit may present users whose exercise available time slots are shifted from that of another user so that the plurality of users can exercise in sequence.

Exercise content may be presented according to the exercise available time slots.

The user information indicating an exercise level or a physical level of the user may be acquired and another user of the same level may be presented.

The user information indicating an exercise level or a physical level of the user may be acquired and an exercise volume difference may be presented according to a difference in the exercise level and the physical level from those of another user.

A program causes a computer to execute a processing method including:
acquiring schedule information indicating schedules of a plurality of users;
extracting exercise available time slots from the schedules of the users; and
comparing the exercise available time slots of the plurality of users and presenting users who can exercise together and corresponding time slots.

According to the present disclosure, it is possible to provide a processing system, a processing method, and a program capable of appropriately motivating a user to exercise.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a control block diagram schematically showing a configuration of a processing system;

FIG. 2 is a table schematically showing schedules of users 1 to 3;

FIG. 3 is a flowchart showing a processing method; and

FIG. 4 is a flowchart showing the processing method.

### DESCRIPTION OF EMBODIMENTS

Hereinafter, the present disclosure will be described through an embodiment of the disclosure, but the claimed disclosure is not limited to the following embodiment. Furthermore, not all of the configurations described in the embodiment are essential as means for solving problems.

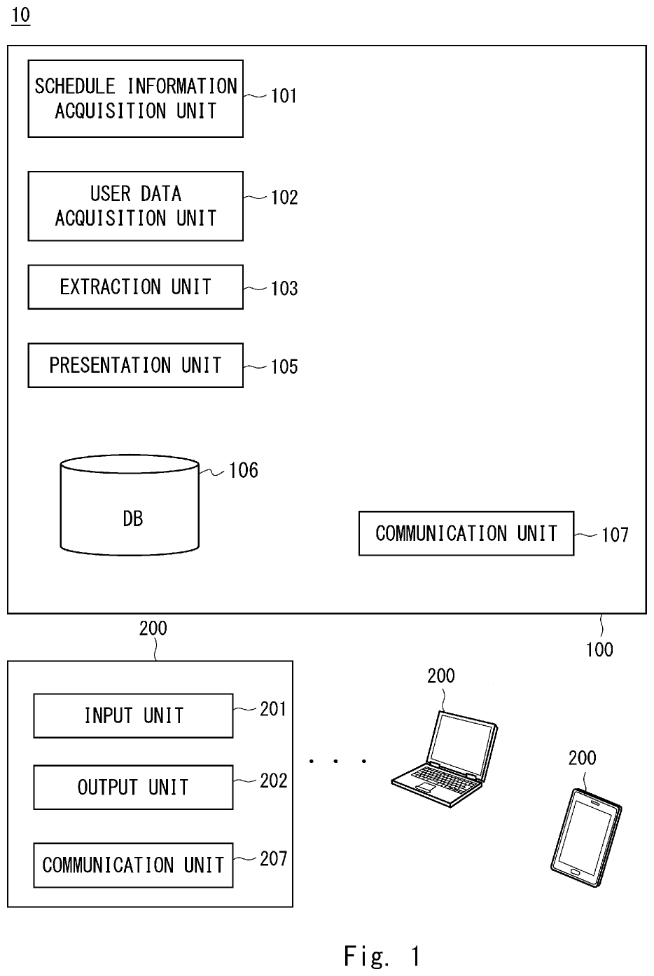

A processing system and a processing method according to the embodiment will be described with reference to the drawings. FIG. 1 is a block diagram showing a configuration of a processing system 10. The processing system 10 includes a processing apparatus 100 and a plurality of terminals 200. The processing system 10 is a recommendation system for recommending exercises to users.

Each of the terminals 200 is a device used by a user. Typically, the terminal 200 is an information processing apparatus such as a personal computer, smartphone, or tablet computer. The terminal 200 may also be a wearable device such as a smartwatch or smart glasses. The terminal 200 may be a combination of a plurality of devices. The terminal 200 is connected to the processing apparatus 100 via a network (not shown). The connection between the terminal 200 and the processing apparatus 100 can be either wired or wireless.

The terminal 200 includes an input unit 201, an output unit 202, and a communication unit 207. The terminal 200 includes a memory for storing programs and the like, and a processor for executing programs and the like. Thus, the terminal 200 executes the following processing by executing the computer programs. The terminal 200 may be associated with user ID information about a user who uses it. The terminal 200 can attach the user ID information to various information and transmit it to the processing apparatus 100. Additionally, the terminal 200 may have a memory or the like for recording user information.

The input unit 201 accepts an input operation of the user. The input unit 201 has an input device such as a touch panel, a keyboard, and a mouse. Various information can be input by the user operating the input device. Of course, the input unit 201 may have a microphone or the like for voice input. Alternatively, the input unit 201 may have, for example, a camera or various sensors for capturing and inputting images of the user.

The user may register a schedule using the input unit 201. The user may input the user information using the input unit 201. The user may input exercise information related to a desired exercise using the input unit 201.

The output unit 202 includes a display device such as a liquid crystal display. By displaying information on a monitor, the output unit 202 can output various information to the user. The output unit may have a speaker, headphones, or the like for audio output. The input unit 201 and the output unit 202 may be a touch panel display or the like. The output unit 202 outputs presentation information related to the exercise to the user. Thus, the exercise can be recommended to the user.

The communication unit 207 has a communication interface with the processing apparatus 100. For example, the communication unit 207 has a transceiver or the like for wireless communication. Various types of information input by the input unit 201 are transmitted to the processing apparatus 100. When the communication unit 207 receives the information from the processing apparatus 100, the output unit 202 outputs the received information to the user.

The processing apparatus 100 functions as a recommendation apparatus for recommending exercises to the user. The processing apparatus 100 includes a schedule information acquisition unit 101, a user information acquisition unit 102, an extraction unit 103, a presentation unit 105, a DB (database) 106, and a communication unit 107. The communication unit 107 includes a communication IF for communicating with each terminal 200. The communication unit 107 receives user information, schedule information, and exercise information from the terminal 200. The communication unit 107 also transmits presentation information to the terminal 200. Since a known technique can be used for data communication processing between the communication unit 107 and the communication unit 207, description thereof will be omitted.

The DB (database) 106 functions as a storage unit for storing the schedule information, the user information, and so on. The DB 106 stores the schedule information and the user information for each user.

The schedule information acquisition unit 101 acquires the schedule information indicating the user's schedule. When each user registers his/her schedule from the terminal 200, the schedule information acquisition unit 101 acquires the schedule information for each user. For example, the schedule information acquisition unit 101 records the schedule information associated with a user ID in the DB 106. The DB 106 stores the schedule information for each user.

The schedule information includes data such as a user ID, a schedule item, a start date and time, and an end date and time. Schedule items represent the content of the schedule, such as individual work (desk work, etc.), meetings, online meetings, travel, outings, visitors, meals, and sleep. In other words, the user's schedule is categorized based on its content. The terminal 200 and the processing apparatus 100 have pre-registered information indicating the categorized schedule items. The schedule information acquisition unit 101 acquires the schedule information from the terminal 200. The terminal 200 may also detect the schedule information. For example, by using a wearable device, the user's schedule can be appropriately detected.

The user information acquisition unit 102 acquires the user information related to the user. The user information includes data about the user's physical level and exercise level. For example, the user information indicating the physical level includes the user's height, weight, age, gender, body fat percentage, and so on. Further, the user information indicating the exercise level includes, for example, the user's exercise frequency, history of exercise content, exercise volume (amount of exercise), marathon completion time, and so on. The user information acquisition unit 102 associates the user information with the user ID and records it in the DB 106. The DB 106 stores the user information for each user.

The user may input the user information by operating the input unit 201. Alternatively, the terminal 200 may automatically detect the user information. For example, by using a wearable device, the user information such as the exercise frequency can be appropriately detected. The user information acquisition unit 102 acquires the user information from the terminal 200.

The extraction unit 103 extracts the time slots during which the user can exercise (such time slots are hereinafter referred to as exercise available time slots) from the user's schedule. That is, the extraction unit 103 extracts the exercise available free time (spare time) from the user's schedule. Here, the exercise available time slot is set as a time of 15 minutes or more. The available exercise content is set for each categorized item of the schedule.

The user can perform a foot-pedaling exercise and walking as exercises. The user can perform a foot-pedaling exercise using a foot-pedaling exercise machine or a bicycle. For example, a foot-pedaling exercise machine may be installed under the user's desk. The user may perform a foot-pedaling exercise during desk work or online meetings. Furthermore, the user can perform a foot-pedaling exercise when he/she moves by bicycle.

Furthermore, the user can perform walking exercises using step-type foot-pedals, treadmills, and other walking exercise machines. Alternatively, the user can perform walking exercises when moving on foot.

The foot-pedaling exercise is defined as an exercise A and walking is defined as an exercise B. Of course, the user may perform exercises other than foot-pedaling exercise and walking. The user may perform aerobic exercises other than foot-pedaling exercise and walking. The user may perform weight training using barbells, dumbbells, etc. Alternatively, the user may perform exercises without equipment. For example, the user may perform strength training exercises like push-ups and squats.

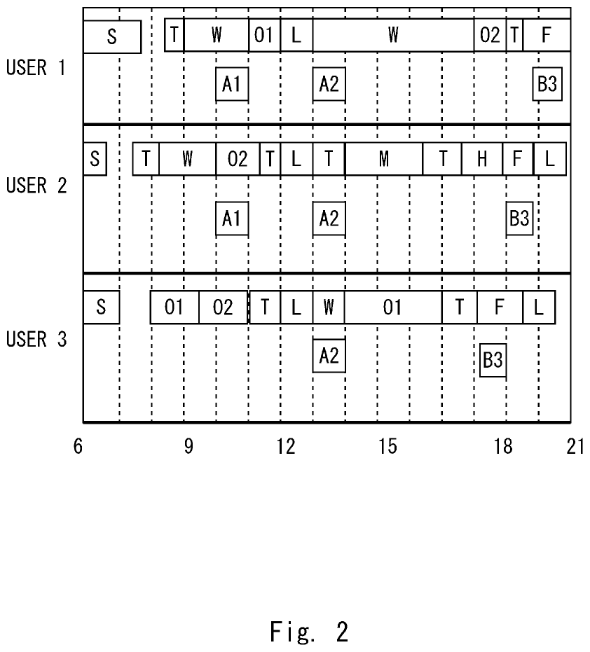

FIG. 2 is a schematic diagram showing schedules of users 1 to 3. The horizontal axis represents time, and FIG. 2 is a timetable from 6:00 in the morning to 9:00 in the evening. In FIG. 2, W represents individual work (desk work, etc.), T represents travel, M represents meetings, F represents free time, L represents meals, S represents sleep, and H represents telecommuting (remote work). Additionally, O1 represents important online meetings, and O2 represents non-important online meetings. The schedule information includes the start and end times for each schedule item.

The free time F is designated as the time available for the exercise A and the exercise B. The meal L, meeting M, and important online meeting O1 are set as times not available for exercises. Additionally, sleep S is also designated as a time not available for exercises.

The travel T is designated as a time available for the exercise A or the exercise B depending on the mode of transportation. For example, if the mode of transportation is a bicycle, T is set as a time available for the exercise A. Alternatively, if the mode of transportation is walking, T is set as a time available for the exercise B. When the user travels by train or car, T is registered as a time not available for exercises. Thus, possible exercises are registered in T depending on the mode of transportation and destination. The extraction unit 103 can set possible exercises in T depending on the destination and mode of transportation. The processing apparatus 100 may specify the mode of transportation depending on the destination. Alternatively, the user may input the mode of transportation.

The individual work W may be set as a time available for the exercise A or the exercise B. For example, if a foot-pedaling machine is installed under the user's desk, he/she can perform the exercise A during the individual work W. If a walking exercise machine is installed under the user's desk, he/she can perform the exercise B during the individual work W. Therefore, the individual work W is registered as a time available for the exercise A or the exercise B. If there is no foot-pedaling or walking exercise machine installed under the user's desk, the individual work W is not registered as a time available for the exercise A or the exercise B.

Similarly, the non-important online meeting O2 is designated as a time available for the exercise A or the exercise B. For example, if a foot-pedaling or walking exercise machine is installed under the user's desk where the user conducts individual work, he/she can perform the exercise A or the exercise B during the non-important online meetings O2. Therefore, the non-important online meeting O2 is registered as a time available for the exercise A or the exercise B. However, if there is no foot-pedaling or walking exercise machine installed under the user's desk, the non-important online meeting O2 is registered as a time not available for the exercise A or the exercise B.

Whether an online meeting is an important online meeting O1 or an unimportant online meeting O2 can be set using various pieces of information. For example, meetings that the user hosts or regular meetings with a lot of speaking are set as the important online meetings O1. Meetings that the user does not host or regular meetings with little speaking are set as the non-important online meetings O2. Alternatively, in the terminal 200, if the camera that captures images of the user is on, the meeting can be set as important, whereas if it is off, it can be set as non-important. The amount of speaking can be determined using the camera or microphone of the terminal 200. For example, the terminal 200 can detect that the user is speaking a lot using the microphone.

Similarly, the telecommuting H is designated as a time available for the exercise A or the exercise B. For example, if a foot-pedaling or walking exercise machine is installed under the user's desk where the user conducts telecommuting H, he/she can perform the exercise A or the exercise B during the x. Therefore, the telecommuting H is registered as a time available for the exercise A or the exercise B. However, if there is no foot-pedaling or walking exercise machine installed under the user's desk, the telecommuting H is not registered as a time available for the exercise A or the exercise B.

The extraction unit 103 extracts the exercise available time slots for each user based on the schedule information. For example, the extraction unit 103 refers to the schedule information of the user 1 to specify the schedule items for which the user 1 can exercise. Then, a time slot in which a schedule item for which the user 1 can exercise is specified is extracted as an exercise available time slot. Furthermore, possible exercises are specified for each schedule item. For the user 1, who has a foot-pedaling machine installed under his/her desk, the individual work W is a time slot when the exercise A is possible.

Each user may have a different correspondence between possible exercise content and schedule items. For example, the exercise A may be possible during the non-important online meetings O2 for the user 2, but not for the user 1. In this way, the correspondence between schedule items and possible exercises is set for each user.

The presentation unit 105 compares the exercise available time slots of a plurality of users and presents the time slots when they can exercise with other users. The user inputs exercise information about a desired exercise using the input unit 201. The exercise information indicates the exercise content. For example, the user inputs the exercise A or the exercise B as a desired exercise. The exercise information may also include the date and time of the exercise. Furthermore, the exercise information may include information about the exercise volume. For example, the target exercise volume, such as a walking distance or calorie consumption, may be included in the exercise information.

The user inputs the exercise information, including the desired exercise date and exercise content, using the input unit 201. The terminal 200 then transmits the exercise information to the processing apparatus 100. The presentation unit 105 generates the presentation information based on the exercise information received by the communication unit 207. The presentation information includes the exercise content, start time, end time, and information about other users who exercise together. Note that among the users who exercise together, the exercise content, start time, and end time may either match those of another user completely or partially. For example, there may be variations in the start and end times among the users.

The communication unit 107 transmits the presentation information to the plurality of terminals 200. Here, the presentation information is transmitted to the plurality of terminals 200 of the users who exercise together. The users of the terminal 200 who receive the presentation information can exercise during the time slot indicated in the presentation information. The plurality of users can exercise together. The presentation information may also include information about the exercise volume. For example, the target exercise volume, such as a walking distance or calorie consumption, may be included in the presentation information. Additionally, the presentation information may include information about the location of the exercise. This allows the plurality of users to exercise in the same location or exercise together remotely from different locations.

For example, if the user 1 inputs the exercise information indicating that he/she wants to do the exercise A in the morning in the schedule shown in FIG. 2. The extraction unit 103 extracts the exercise available time slots, such as the time slot for the individual work W. The presentation unit 105 presents a time slot A1, which is from 10:00 to 11:00 in the morning, as the time slot during which the exercise A can be performed. The presentation unit 105 also presents the user 2 as a user who can exercise together with the user 1. Based on the exercise information, the presentation unit 105 presents the user 2 and the time (10:00 to 11:00) during which he/she can exercise. In this way, the presentation unit 105 presents a user who can exercise together based on the exercise information.

The presentation unit 105 transmits the presentation information to the terminals 200 of the users 1 and 2. The terminals 200 display the time for the exercise A, which is from 10:00 to 11:00 (A1 in FIG. 2), as the time during which the exercise A can be performed. Additionally, the terminals 200 display information about the users 1 and 2, who can exercise together. For example, the processing apparatus 100 transmits user information about the user 2 to the terminal 200 of the user 1. The terminal 200 of the user 1 displays the user information about the user 2, who can exercise together.

The processing apparatus 100 transmits the user information about the user 1 to the terminal 200 of the user 2. The terminal 200 of the user 2 displays the user information about the user 1. Specifically, the terminal 200 displays that there is a guidance (invitation) from the user 1 to exercise together. Of course, exercising together does not necessarily mean exercising in the same physical location. Therefore, the method according to this embodiment can be applied to a plurality of users who exercise in remote locations. The presentation unit 105 can select users who can exercise together from those in remote locations.

Additionally, the exercise content may be the same or different for users exercising together.

In this way, users can be motivated to exercise appropriately. The users can utilize the spare times in their schedules to exercise together with other users. This increases their motivation and allows them to maintain a consistent exercise routine, thereby enhancing the effectiveness of their workouts. The users can also recognize other users who can engage in the same type of exercise, further boosting their motivation. Therefore, it is possible to provide users with the motivation to exercise appropriately. Additionally, the presentation unit 105 can compare the exercise available time slots of a plurality of users within the overall schedule and provide presentations for exercising together. In this case, the users do not need to input the exercise information.

Additionally, the output unit 202 of the terminal 200 may have a monitor displaying the users who are exercising simultaneously. For example, the terminal 200 may have a camera that captures a video of each user while he/she is exercising. When the camera of the terminal 200 or the like captures a video of the user 1 exercising, the terminal 200 transmits the video to the terminal 200 of the user 2. Thus, the user 2 can view the video of the user 1 exercising and can exercise at the same time. The user 2 can exercise while watching the video of the user 1. Similarly, the user 1 can exercise while watching the video of the user 2. This allows the processing system 10 to provide users with a higher level of motivation. Note that the user's video may be the actual video of the user or an avatar-like representation.

Furthermore, the output unit 202 of the terminal 200 may display information that encourages a plurality of users to compete the exercise volume with each other. For example, the terminal 200 of the user 2 displays exercise volume information that indicates the user 1's running distance or speed. The terminal 200 of the user 2 updates the user 1's exercise volume information to the latest value. As a result, the user 2's competitiveness increases, and his/her motivation to exercise can be increased. In other words, the plurality of users can compete with each other as competitors. This lead to a higher level of motivation of the users to exercise.

Furthermore, in the above description, while the presentation unit 105 presents users who can exercise together at the same time, it is also possible to present users who exercise at different time slots. The times when the plurality of users exercise together may overlap or match that of another user. Alternatively, the times when the plurality of users exercise together may be shifted from each other. For example, even if the plurality of users exercise in sequence, the presentation unit 105 can present them as users who can exercise together.

For example, in the schedule shown in FIG. 2, the user 2 inputs the exercise information indicating that the user 2 desires to perform the exercise B between 17:00 and 18:00. The exercise available time slots of the users 1 to 3 are shifted. To allow the users 3, 2, and 1 to perform the exercise B in this order, the presentation unit 105 provides the presentation (B3 in FIG. 2). For instance, the presentation unit 105 presents the time from 17:15 to 18:00 to the user 3. The presentation unit 105 presents the time from 18:00 to 18:45 to the user 2. The presentation unit 105 presents the time from 18:45 to 19:30 to the user 1. The times presented a plurality of users may partially overlap.

In this way, the presentation unit 105 presents other users who have available exercise spare time slots shifted from each other as users who can exercise together. This allows a plurality of users to exercise in sequence. In this case, the plurality of users can perform the exercise B consecutively, giving them a sense of relay or long distance relay race like experience. This enhances the sense of solidarity and provides a higher level of motivation. In such a case, the terminal 200 may display the total running distance and total exercise time of the plurality of users on the monitor. This enables users to engage in exercise with a high level of motivation.

The presentation unit 105 may also present the exercise content based on the exercise available time slots. For example, the presentation unit 105 may present the exercise A using a foot-pedaling exercise machine placed under the desk or the exercise B using a foot stepping machine during desk work, not just during completely free time. Alternatively, the presentation unit 105 may present exercises such as standing on one leg that can be done just by standing near the desk. The exercise content that can be exercised may be set according to the schedule item.

The presentation unit 105 may also present exercise options such as walking or cycling instead of using transportation (such as taxis or trains) during travel time or the like. This can increase the frequency of exercise and make it more effective. Additionally, the presentation unit 105 may present more effective exercise options taking into account meal and sleep times. For example, if high-intensity exercise is to be performed, the presentation unit 105 presents a time slot of 1 to 2 hours after a meal or 3 hours before sleep.

The presentation unit 105 may prompt users to select other users who can exercise together when a plurality of users are available for exercise together. For example, if the user 2 has a time slot for the exercise A (A2 in FIG. 2), both the user 1 and the user 3 become candidates who can exercise together. In this case, the user 2 may select either the user 1 or the user 3 as a user to exercise together with. The terminal 200 of the user 2 or the processing apparatus 100 may transmit the presentation information only to the selected user.

For example, if the user 2 selects the user 1 as the user to exercise together with for the exercise A, the terminal 200 or the processing apparatus 100 notifies the user 1's terminal 200 with a guidance. If the user 2 selects the user 3 as the user to exercise together with for the exercise A, the terminal 200 or the processing apparatus 100 notifies the user 3's terminal 200 with a guidance. If the user 2 selects both the user 1 and the user 3 as the users to exercise together with for the exercise A, the terminal 200 or the processing apparatus 100 notifies both the user 1 and the user 3's terminals 200 with a guidance. In this way, the guidance is transmitted only to the selected users. The user 2 can select one or more users to exercise together with.

Furthermore, the presentation unit 105 may generate the presentation information based on the user information indicating the user's exercise level and physical level. Specifically, the presentation unit 105 may present users with similar exercise levels and physical levels. This can increase competitiveness and enhance motivation among the users, leading to improved exercise performance.

For example, if the user 2 inputs the exercise information indicating that he/she desires to perform the exercise A in the afternoon, the presentation unit 105 presents the time slot A2 from 13:00 to 14:00. At this time, both the user 1 and the user 3 can perform the exercise A together. Additionally, the level of the user 3 is closer to that of the user 2 than that of the user 1. In this case, the presentation unit 105 presents the user 2 with the user 3 as a user who can exercise together.

Of course, the presentation unit 105 may also present all users who can exercise together. In this case, the presentation unit 105 may display the users along with their respective levels. The presentation information may include the user's level. The users can choose a user to exercise together from among a plurality of candidates. For example, a user may select a user with a similar level to exercise together with. Alternatively, a user may choose to exercise with a user at a higher level.

Furthermore, the presentation unit 105 may also present differences in exercise volume based on a difference between the levels of users when presenting the users with different exercise levels. For instance, for users with lower exercise levels or physical levels, the presentation unit 105 may provide handicaps based on the difference between the levels of users. Further, for users with higher exercise levels or physical levels, the presentation unit 105 may increase the distance to the goal for running activities or increase the exercise machine load.

The presentation unit 105 may acquire the user information indicating the user's exercise level and physical level to present a difference in the exercise volume based on the difference between the levels of users. The presentation unit 105 may adjust the exercise volume based on the difference between the levels of users. This can increase competitiveness even of users among whom there is a large level gap. The processing system 10 can thus provide users with a higher level of motivation.

Furthermore, the terminal 200 may detect the actual exercise volume of the performed exercises. The processing apparatus 100 may then acquire exercise volume data from the plurality of terminals 200 and incorporate the feedback results into presentation processing. Specifically, the processing apparatus 100 may allow a learning model to be trained to recognize which combinations of individuals lead to the performing of continued exercise. This enables the presentation of the most suitable users for exercising together.

Furthermore, the presentation unit 105 can present users who get along well or those who stimulate a sense of competition. Furthermore, the presentation unit 105 can provide presentations tailored to a user's personality. The machine learning model can estimate information such as whether an exercise can be continued when individuals have similar exercise levels. The machine learning model can also estimate whether the user can continue exercising when a user exercises together with another user who has a higher exercise level than the user himself/herself or with another user who has a lower exercise level than the user himself/herself. This allows users to continue exercising for longer periods. For example, the machine learning model may learn whether users of the same gender or opposite gender are more likely to continue exercising together.

In this way, the processing apparatus 100 can utilize the user information to perform machine learning. The machine learning model can use the user information to provide presentations. Alternatively, the processing apparatus 100 can conduct machine learning using feedback and the like received from the users. During or after the exercise, the terminal 200 may inquire whether a user desires to exercise again with the same other user. When the user responds to the inquiry, the results are fed back to the processing apparatus 100. By doing so, more suitable users can be presented.

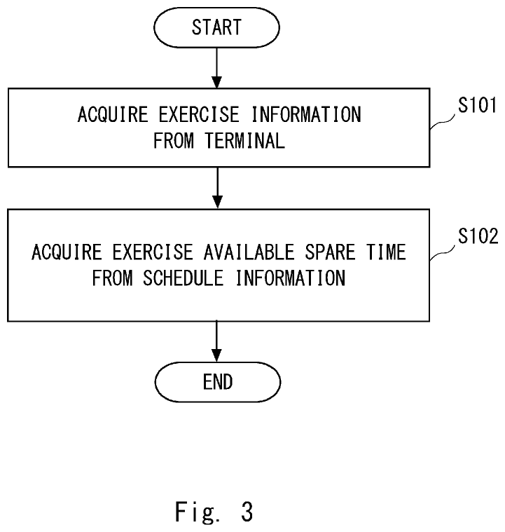

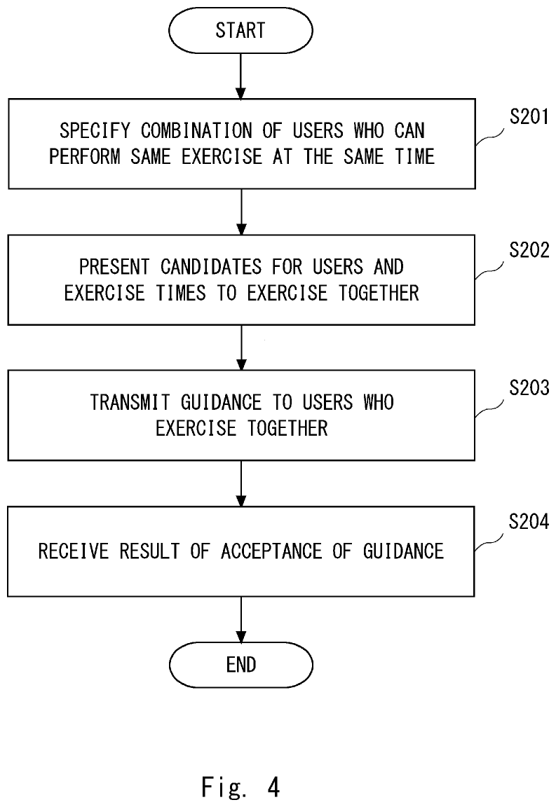

Next, a presentation method according to this embodiment will be described with reference to FIGS. 3 and 4. FIGS. 3 and 4 are flowcharts showing a presentation method.

First, the presentation unit 105 acquires the exercise information from the terminal 200 (S101). For instance, the user 1 operates the input unit 201 to input the exercise A that he/she wants to perform during his/her spare time. The communication unit 207 transmits the exercise information indicating the exercise content to the processing apparatus 100. This allows the presentation unit 105 to acquire the exercise information. Here, the user can specify not only the exercise content but also the date, day of the week, and time slot for performing the exercise. For example, the user can input that he/she wants to do the exercise A every Thursday afternoon. The user 1 may also input his/her target exercise volume. In this case, the presentation unit 105 acquires the exercise information that includes both the time and exercise volume. Of course, the user can input only the exercise content.

Next, the presentation unit 105 acquires the available exercise spare time from the schedule information (S102). For example, in the DB 106, the schedule information is pre-registered for each user. The presentation unit 105 extracts the available time slot for performing the exercise A from the user 1's schedule information. Of course, the presentation unit 105 can extract two or more spare times.

As shown in FIG. 4, the presentation unit 105 specifies users who can perform the same exercise at the same time (S201). In other words, the presentation unit 105 refers to the schedule information in the DB to specify users who can perform the exercise A during the spare time. The presentation unit 105 extracts one or more users whose spare times match that of another user. Of course, the spare times may be at the exact same time or may have some time gap between them. If there are two or more spare times, the extraction unit 103 extracts data for each of these spare times. For example, the presentation unit 105 may specify the user 2 as a user with whom the user 1 can exercise together during the time from 11:00 to 12:00 and specify the user 3 as a user with whom the user 1 can exercise together during the time from 13:00 to 14:00.

The presentation unit 105 presents candidates for users and exercise times to perform the exercise A together (S202). The presentation unit 105 generates the presentation information including users and times, and the communication unit 107 transmits the presentation information to the user 1's terminal 200. The output unit 202 displays the users and times included in the presentation information. This allows the user 1 to review the presentation information and select the user to perform the exercise A together and the time. The user 1 can select a user to exercise with from among the plurality of candidates. In the following example, it is assumed that the user 1 uses the input unit 201 to select performing the exercise A together with the user 2 from 11:00 to 12:00.

Next, the terminal 200 transmits a guidance to the selected user to exercise together (S203). When the user 1 selects the user 2 to exercise together, the terminal 200 transmits the guidance to the selected user. In the above example, the user 1's terminal 200 transmits the guidance to the user 2's terminal 200. The guidance includes the presentation information indicating the user 1 and the exercise time. The presentation information includes the exercise time of, for example, 11:00 to 12:00. The guidance to the user 2 includes the presentation information about exercising together with the user 1 and the specified time therefor. Note that the guidance may instead be transmitted via the processing apparatus 100.

The user 1 receives a result of acceptance of the guidance (S204). The user 2 operates the input unit 201 so that exercising together with the user 1 is accepted. The user 2's terminal 200 transmits a result of acceptance of the guidance to the user 1's terminal 200. The user 1 is thus made aware of the fact that the guidance has been accepted. As a result, the user 1 and the user 2 can exercise together for the exercise A. This enables the users to be appropriately motivated, thus allowing them to continue exercising and enhancing the effectiveness of the exercise. If the user 2 does not accept exercising together with the user 1, the presentation unit 105 may present other users or alternative exercise times.

In this way, the processing apparatus 100 and the processing system 10 can provide the user with an appropriate motivation to perform exercise.

The processing apparatus 100 or the processing system 10 may be implemented by one or more computers. For example, the processing apparatus 100 may include a plurality of apparatuses for distributed processing. The processing system 10 is typically an information processing apparatus such as a personal computer or a smartphone. Specifically, the processing system 10 includes a memory for storing programs and the like and a processor for executing the programs and the like. The processing system 10 may include a plurality of processors for performing distributed processing. For example, a server apparatus connected by a network and a user terminal may cooperate to perform processing.

Some or all of the above processing may be executed by a computer program. That is, a control computer constituting the processing system 10 executes the program to control the processing system 10. The program described above includes instructions (or software code) for causing the computer to perform one or more functions described in the embodiment when read into the computer.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A processing system comprising:
a schedule information acquisition unit configured to acquire schedule information indicating schedules of a plurality of users;
an extraction unit configured to extract exercise available time slots from the schedules of the users; and
a presentation unit configured to compare the exercise available time slots of the plurality of users and present users who can exercise together and corresponding time slots.

2. The processing system according to claim 1, further comprising a monitor configured to display the users performing exercises simultaneously.

3. The processing system according to claim 2, wherein the presentation unit presents users whose exercise available time slots match that of another user so that the plurality of users can exercise simultaneously.

4. The processing system according to claim 1, wherein the presentation unit presents users whose exercise available time slots are shifted from that of another user so that the plurality of users can exercise in sequence.

5. The processing system according to claim 1, wherein the presentation unit provides exercise content according to the exercise available time slots.

6. The processing system according to claim 1, wherein the user information indicating an exercise level or a physical level of the user is acquired and another user of the same level is presented.

7. The processing system according to claim 1, wherein the user information indicating an exercise level or a physical level of the user is acquired and an exercise volume difference is presented according to a difference in the exercise level and the physical level of the user from those of another user.

8. A processing method comprising:
acquiring schedule information indicating schedules of a plurality of users;
extracting exercise available time slots from the schedules of the users; and
comparing the exercise available time slots of the plurality of users and presenting users who can exercise together and corresponding time slots.

9. The processing method according to claim 8, further comprising displaying the users performing exercises simultaneously on a monitor.

10. The processing method according to claim 9, wherein users whose exercise available time slots match are presented so that the plurality of users can exercise simultaneously.

11. The processing method according to claim 8, wherein users whose exercise available time slots are shifted from that of another user are presented so that the plurality of users can exercise in sequence.

12. The processing method according to claim 8, wherein exercise content is presented according to the exercise available time slots.

13. The processing method according to claim 8, wherein the user information indicating an exercise level or a physical level of the user is acquired and another user of the same level is presented.

14. The processing method according to claim 8, wherein the user information indicating an exercise level or a physical level of the user is acquired and an exercise volume difference is presented according to a difference in the exercise level and the physical level of the user from those of another user.

15. A non-transitory computer readable medium storing a program for causing a computer to execute a processing method comprising:
acquiring schedule information indicating schedules of a plurality of users;
extracting exercise available time slots from the schedules of the users; and
comparing the exercise available time slots of the plurality of users and presenting users who can exercise together and corresponding time slots.
